(19) United States
(12) Patent Application Publication
(10) Pub. No.: US 2008/0262529 A1
(43) Pub. Date: Oct. 23, 2008

(54) Gastric Balloon Devices and Methods of Use

(75) Inventor: Steven L. Jacques, Westford, MA (US)

Correspondence Address: HOLLAND & HART LLP, 60 E. South Temple, Suite 2000, P.O. Box 11583, Salt Lake City, UT, 84110, US

(73) Assignee: C.R. Bard, Inc., Murray Hill, NJ (US)

(21) Appl. No.: 11/596,234

(22) PCT Filed: May 12, 2005
(86) PCT No.: PCT/US05/16736

§ 371 Date: Nov. 13, 2006

Related U.S. Application Data
(60) Provisional application No. 60/571,086, filed on May 14, 2004.

(51) Int. Cl. A61M 29/02

(52) U.S. Cl. 606/192; 606/191

(57) ABSTRACT

A gastric balloon devices and systems (8) for occupying space within a stomach of a patient are disclosed. In one embodiment, such a gastric balloon device (8) includes a shaft element (14) for insertion into a stomach (52) of a patient and a generally elongated balloon element (12) having at least one pleat region (30). Methods of use of a gastric balloon device or system are also disclosed. For example, a gastric balloon system (8) is provided including a generally elongated balloon element (12), wherein the balloon element (12) includes at least one pleat region (30). Further, the balloon element (12) is furled and inserted through a stoma into the stomach (52) of the patient and at least partially expanded. In another method a balloon element (12) is inserted into a stomach (52) of a patient, wherein a stoma is formed nearer to a duodenum (55) than a fundus region of the stomach (52), and the balloon element (12) is at least partially expanded.

### CROSS REFERENCE TO RELATED APPLICATION

This application claims the benefit of the filing date of U.S. Provisional Patent Application No. 60/571,086, filed May 14, 2004, the disclosure of which is incorporated, in its entirety, by this reference.

### BACKGROUND OF THE INVENTION

Severe or “morbid” obesity is a complex, medical disease, which affects more than nine million people in the United States. Studies have shown that diets, medications, behavioral modification or exercise programs have an extremely high failure rate in this population, due to underlying physiologic, chemical, and genetic factors. Losing excess weight is very important for improving health and well-being.

Medical dietary regimes and behavior modification are commonly used as an initial treatment of obesity since they have almost no side effects or complications, when properly applied and monitored. However, these methods are usually unsuccessful in the treatment of morbid obesity because they may depend largely upon the willpower of the patients. Other methods of treatment including devices and surgical procedures, which may also be employed in combination with behavior modification and medical diets.

Surgical procedures for treatment of obesity include procedures that lead to weight loss include gastric bypass surgery, gastroplasty, gastric stapling, and oral surgical procedures such as wiring shut the patient's jaws to reduce food intake. These procedures are usually relatively effective in producing weight loss but some of them have been accompanied by serious complications and side effects, including operative mortality, postoperative wound infection, liver disfunction and failure, kidney stones, diarrhea, or further surgeries to treat intestinal obstruction or hernias or to revise original surgery.

### SUMMARY OF THE INVENTION

The present invention contemplates a gastric balloon devices and systems for occupying space within a stomach of a patient. In one embodiment, such a gastric balloon device includes a shaft element for insertion through a stoma formed in a stomach of a patient and a generally elongated balloon element sealed to and extending from the shaft element and having at least one pleat region. As described herein, many configurations are contemplated for the at least one pleat region of the balloon element. For example, one embodiment of a balloon element includes four pleat regions arranged along substantially perpendicular axes. In another example, one embodiment of a balloon element includes three pleat regions arranged so as to form substantially equal angles therebetween.

The present invention also contemplates methods of use of a gastric balloon device or system according to the present invention. Particularly, in one embodiment, a gastric balloon system is provided including a shaft element for insertion through a stoma formed in a stomach of a patient and a generally elongated balloon element sealed to and extending from the shaft element, wherein the balloon element includes at least one pleat region. Further, the at least one pleat region of a balloon element is furled and inserted through a stoma into the stomach of the patient. The balloon element may be expanded.

In another method of use according to the present invention, a balloon element is inserted through a stoma into a stomach of a patient, wherein the stoma is formed nearer to a duodenum of the stomach than a fundus region of the stomach, and the balloon element is expanded.

The features, advantages, and alternative aspects of the present invention will be apparent to those skilled in the art from a consideration of the following detailed description taken in combination with the accompanying drawings.

### BRIEF DESCRIPTION OF THE DRAWINGS

FIG. 1 shows a schematic side cross-sectional view of a gastric balloon device according to the present invention;

FIG. 2 shows an enlarged, partial view of the gastric balloon device shown in FIG. 1;

FIG. 3 shows a side cross-sectional view of one embodiment of a balloon element as shown in FIGS. 1 and 2;

FIG. 4 shows a side cross-sectional view of another embodiment of a balloon element as shown in FIGS. 1 and 2;

FIG. 5 shows a side cross-sectional view of a further embodiment of a balloon element as shown in FIGS. 1 and 2;

FIG. 6A shows a side cross-sectional view of an additional embodiment of a balloon element as shown in FIGS. 1 and 2;

FIGS. 6B-6D show, in a side cross-sectional view, additional embodiments of a balloon element having six pleat regions, five pleat regions, and eight pleat regions, respectively.

FIG. 7 shows a schematic side view of gastric balloon device according to the present invention in an at least partially expanded state;

FIG. 8 shows an end view of the gastric balloon device shown in FIG. 7;

FIG. 9 shows a schematic side view of gastric balloon device according to the present invention in an at least partially expanded state and including an arcuately shaped region;

FIG. 10 shows an enlarged view of a portion of a balloon element including a coating;

FIG. 11 shows a side schematic view of one embodiment of a gastric balloon system according to the present invention configured for insertion within a patient;

FIG. 12 shows a side cross-sectional view of the gastric balloon system shown in FIG. 11;

FIG. 13 shows a schematic view of a gastric balloon device positioned within a stomach of a patient;

FIG. 14 shows an enlarged view of the gastric balloon element of the gastric balloon device shown in FIG. 14;

FIG. 15 shows a schematic cut-away perspective view of a gastric balloon device implanted within a stomach of a patient;

FIG. 16 shows a schematic view of a gastric balloon device according to the present invention implanted within a stomach of a patient;

FIG. 17 shows a schematic view of a gastric balloon device according to the present invention implanted within a stomach of a patient; and

FIG. 18 shows a schematic view of a gastric balloon device according to the present invention implanted within a stomach of a patient.

### DETAILED DESCRIPTION OF THE INVENTION

The present invention may provide a minimally invasive and relatively safe procedure and device (for initial placement or replacement) for treatment of obesity. For example, severely obese patients, moderately obese patients, or overweight people who suffer from type 2 diabetes or life-threatening cardiopulmonary problems (e.g., severe sleep apnea or obesity-related diseases) may beneficially employ a device according to the present invention. The present invention may also be practiced in connection with any other situation in which weight loss is prescribed or otherwise desirable.

Generally, the present invention contemplates that a gastric balloon may be positioned within a stomach of a patient via endoscopic observation. Further, such a gastric balloon device may be at least partially filled with a fluid, such as saline, water, air, or mixtures thereof. The balloon may be any suitable balloon device that is appropriate for implementing the features of the present invention, as described hereinbelow in greater detail. Thus, a balloon element may be positioned within a stomach and subsequently at least partially expanded so as to occupy space (e.g., volume displacement may be approximately ⅔ of a maximum stomach volume) therein. Such volume displacement may produce a feeling of fullness within a patient and, as a result, a patient may be satisfied with smaller volumes of food, which may result in weight loss.

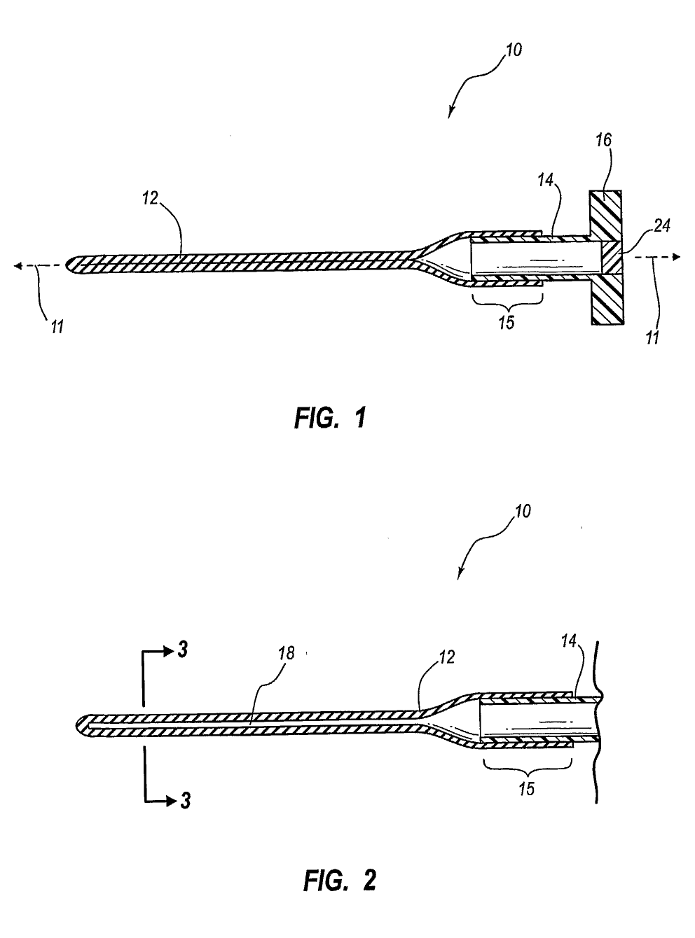

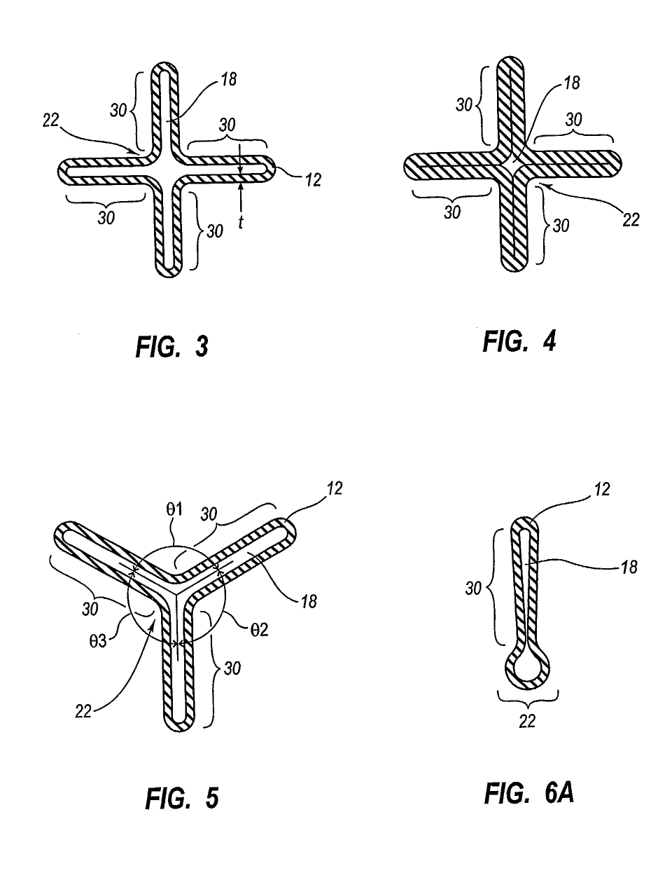

FIG. 1 shows a schematic side cross-sectional view of a gastric balloon device 8 of the present invention including a balloon element 12 sealed to a shaft element 14. Balloon element 12 may be sealed to shaft element such that fluid communication with an interior or lumen of shaft element 14 may cause the balloon element 12 to expand or inflate. As shown in FIG. 1, balloon element 12 may overlap with and be affixed to shaft element 14 within region 15. Further, a valve element 24 may be operably coupled to shaft element 14 and configured for controlling the introduction and release of fluids (e.g., air, liquid, or mixtures thereof) to or from balloon element 12 via shaft element 14. In one embodiment, valve element 24 may comprise a valve that allows for a hollow slender member, such as a needle, to pass through for fluid communication with the balloon element 12, and which reseals when the hollow slender member is withdrawn. Such valves may be found within balls for playing sports, such as soccer or basket balls. In another embodiment, a septum such as a septum found in an access port may be employed as valve element 24. In another embodiment, a so-called needleless access port may be employed as valve member 24. Of course, many valve structures for controlling fluid communication are known in the art and may be utilized as valve member 24, without limitation. As shown in FIG. 1, balloon element 12 may extend in a generally elongated (i.e., generally along longitudinal axis 11) fashion from shaft element 14 and may be substantially compact or closely folded in an unexpanded state, as discussed in further detail hereinbelow. Shaft element 14 may comprise a tubular element as known in the art. For example, in one embodiment, shaft element 14 may comprise a 20 Fr. feeding tube, which may optionally be reinforced, as known in the art. Of course, any suitable catheter or feeding tube as known in the art may comprise shaft element 14, without limitation. The balloon element 12 may have a thickness (FIG. 3) of about 0.015 inches to about 0.030 inches thick, although any suitable thickness may be used. As discussed in greater detail below, a balloon element of the present invention may have a thickness that exceeds a typical thickness for conventional gastric balloon devices. The balloon element 12 may be expanded to any suitable volume as may be desired. For example, one suitable range of volume to which the balloon element may be expanded is about 400 cubic centimeters to about 800 cubic centimeters. As may be appreciated, such a range is merely illustrative and that any other suitable volume or volume range may be accommodated by the present invention. Further, gastric balloon device 8 may include a bolster 16 coupled to the shaft element 14, wherein the bolster 16 is sized and configured for retaining a position of the gastric balloon device 8 when it is positioned within a patient. Bolster 16 may be a so-called 90° bolster, as known in the art, a so-called linear bolster, as known in the art, or any other type of external bolster as known in the art, without limitation. The shaft element 14, balloon element 12, or bolster 16 may comprise a suitable material (e.g., a polymer or metal), as known in the art. For example, the above-described components (e.g., the shaft element 14, balloon element 12, and bolster 16) may comprise silicone. In another embodiment, such components may be made of biocompatible polyurethane or any other suitable material. It should be understood that the above-listed components may each comprise a different material or materials, if desired, without limitation.

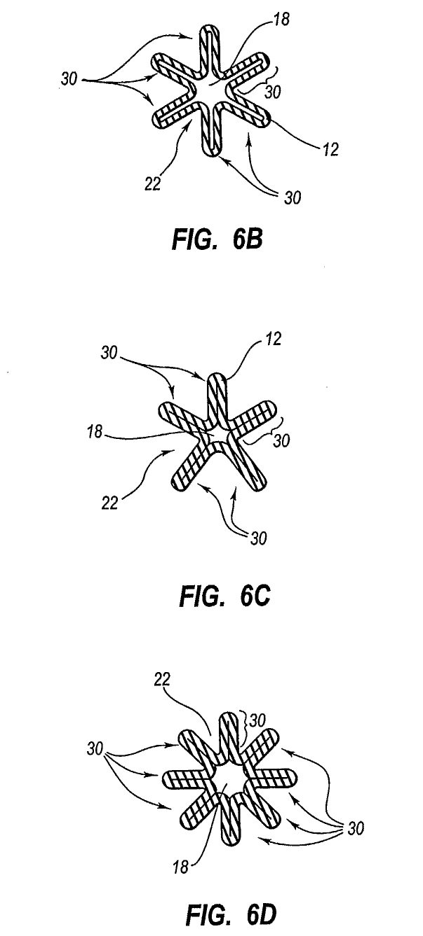

FIG. 2 shows an enlarged, partial view of the gastric balloon device 8 shown in FIG. 1. Particularly, FIG. 2 illustrates that a chamber 18 may exist within an interior of balloon element 12. Of course, the size of such a chamber 18 may depend largely on the pressure of a fluid therein. In another aspect of the present invention, the present invention contemplates that the balloon element 12 may include at least one pleated or folded region extending generally transverse to the longitudinal axis 11. More particularly, FIG. 3 shows a side cross-sectional view taken along reference line A-A, as shown in FIG. 2, of one embodiment of a balloon element 12. As shown in FIG. 3, balloon element 8 may include four pleat regions 30 extending generally from a central region 22. Further, as shown in FIG. 3, pleat regions 30 may be oriented along generally perpendicular axes. As mentioned above, a size of chamber 18 may be influenced by a pressure therein. However, it should also be appreciated that a size of chamber 18 may be designed for exhibiting a selected volume. For example, it may be desirable to generally minimize or reduce the size of chamber 18 so that an overall size of the balloon element 8 may be correspondingly reduced. FIG. 4 shows a balloon element 8 which is structured generally as described with respect to FIG. 3, but wherein chamber 18 is substantially limited to the central region between each of the pleat regions 30. In one example, such a chamber 18 may be formed by generating a vacuum within a balloon element 8 as shown in FIG. 3. In another example, such a chamber 18 having substantially ambient atmospheric pressure therein (i.e., balloon element 8 may be designed so as to occupy a generally minimal cross-sectional space, when unexpanded. More generally, the present invention contemplates that a balloon element of the present invention may include at least one pleat region. Accordingly, in another embodiment, FIG. 5 shows a side cross-sectional view of one embodiment of a balloon element 12 having three pleat regions 30 that extend from central region 22. As shown in FIG. 5, pleat regions 30 may be spaced from one another such that angle θ1, θ2, and θ3 are substantially equal to one another (i.e., about 120°). FIG. 6A shows a side cross-sectional view of another embodiment of a balloon element 12 having three one region 30 that extend from central region 22. As shown in FIG. 6A, pleat region 30 may extend from a generally circular central region 22. A balloon element including at least one pleat region may be advantageous because such a configuration may provide a structure that is more easily removed from (or inserted within) a stomach of a patient. Particularly, during removal of a gastric balloon device of the present invention, the at least one pleat of the balloon element may be structured for furling or otherwise rolling or bending around a central region or longitudinal axis of the balloon element and, therefore, may allow for ease of removal from a stomach of a patient. Further, because the balloon element may be more easily removed from a patient, the thickness of the balloon may be increased (e.g., about 0.015 inches to about 0.030 inches), which will provide a gastric balloon device that does not require replacement as frequently as other gastric balloon devices that have balloon elements with a lesser balloon thickness.

It should be understood, as mentioned hereinabove, that the present invention contemplates any number or pleat regions may be included by a balloon element according to the present invention. Thus, FIGS. 6B-6D each show an additional embodiment of a balloon element 12 including six pleat regions 30, five pleat regions 30, and eight pleat regions 30, respectively. It should be understood that there are many variations to the number, size, and configuration of pleat regions that may be included by a balloon element according to the present invention. Further, some pleat regions may be configured differently than other pleat regions of a balloon element according to the present invention. Thus, the present invention contemplates that a single pleat region or a plurality of pleat regions may be included by a balloon element according to the present invention, without limitation.

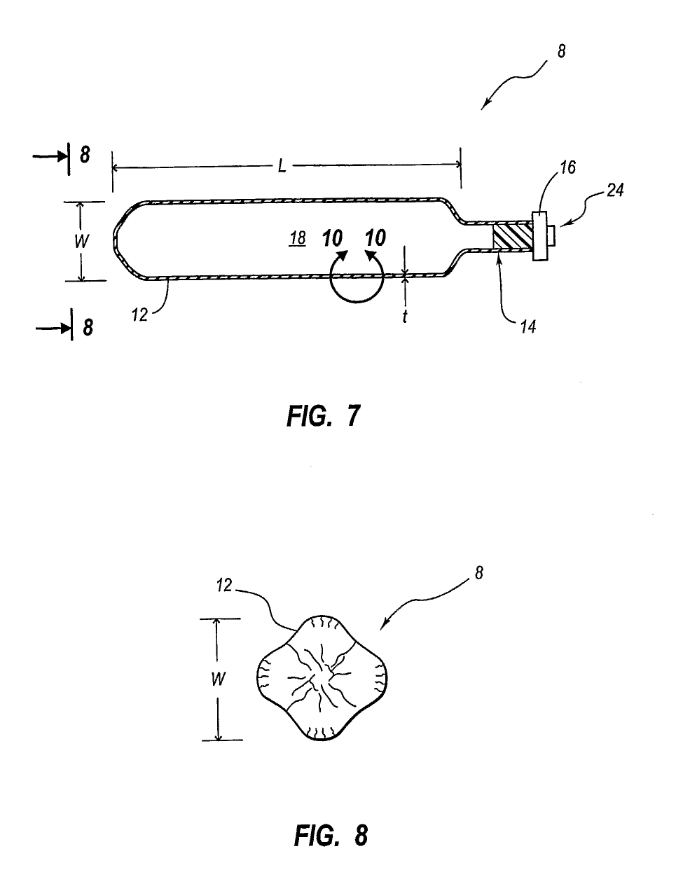

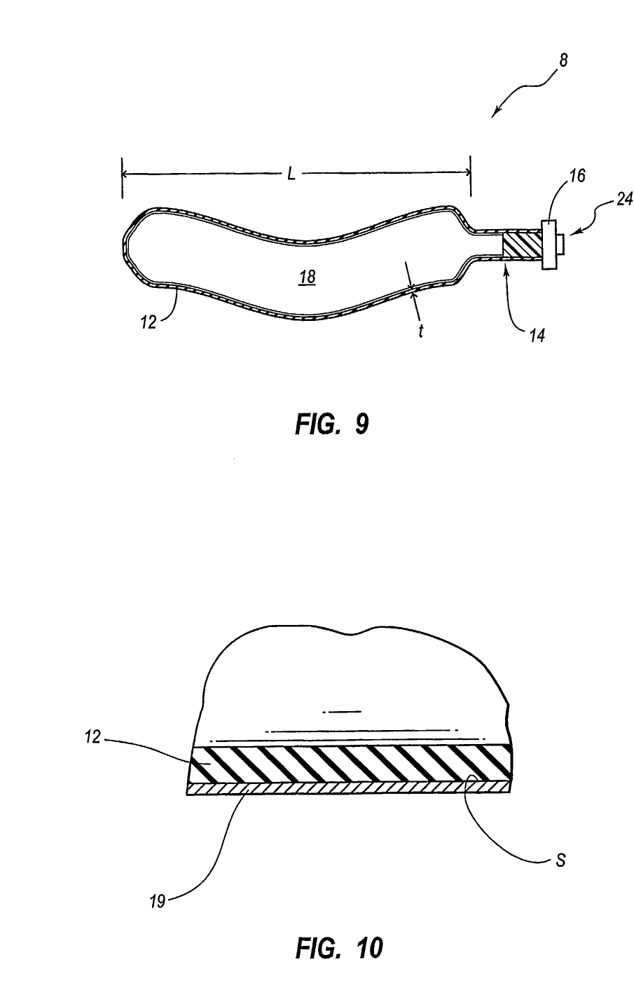

In addition, a balloon element of the present invention may be expanded by introducing a fluid therein. More specifically, FIG. 7 shows a schematic view of gastric balloon device 8 in an at least partially expanded state and exhibiting a length of L and a width W. In addition, FIG. 8 shows an end view of gastric balloon device 8 taken from reference line B-B. Explaining further, a fluid may be introduced within chamber 18 of balloon element 12 via valve element 24. In response, balloon element 12 may expand, according to the materials, shape, and size thereof. Of course, the contents of balloon element 12 may be removed therefrom via valve element 24 so as to collapse chamber 18. The present invention also contemplates that a balloon element 12 may be structured so that expansion thereof causes the balloon element 8 to assume a selected shape. For example, FIG. 9 shows a schematic view of a gastric balloon device 8 having a balloon element 12 that is at least partially expanded. As shown in FIG. 9, at least a portion of balloon element 12 (generally along longitudinal axis 11) may be generally arcuately shaped. For example, at least a portion of balloon element 12, when at least partially expanded, may be substantially circular, substantially elliptical, or otherwise arcuately shaped. Such a configuration may be desirable for providing a balloon element that, when expanded, generally corresponds to a shape of a stomach in which it is positioned, as explained in further detail hereinbelow.

In a further aspect of the present invention, a coating may be formed on at least a portion of an exterior surface of the balloon element 12 for enhancing an aspect of the performance thereof. For example, FIG. 10 shows an enlarged view of a portion of a balloon element 12 including a coating 19. As shown in FIG. 10, coating 19 may be formed upon at least a portion of an exterior surface S of the balloon element 12. In one embodiment, coating 19 may be formulated to be lubricious. For example, coating 19 may be hydrophilic. Such a configuration may ease removal or insertion of the balloon element 12 from or to, respectively, a stomach of a patient. In another embodiment, coating 19 may be formulated for exhibiting resistance to a stomach acid or other chemicals found in the stomach. In one example, coating 19 may comprise at least one of the following: Cellacefate, Cellulosi acetas phthalas, and Cellacefate. Further, coating 19 may be formed over substantially the entire exterior surface S of the balloon element 12 or a portion thereof, without limitation. Such a configuration may allow a gastric balloon device 8 according to the present invention to remain within a stomach for a longer period of time without being replaced. Further, coating 19 may comprise an anti-microbial coating as known in the art.

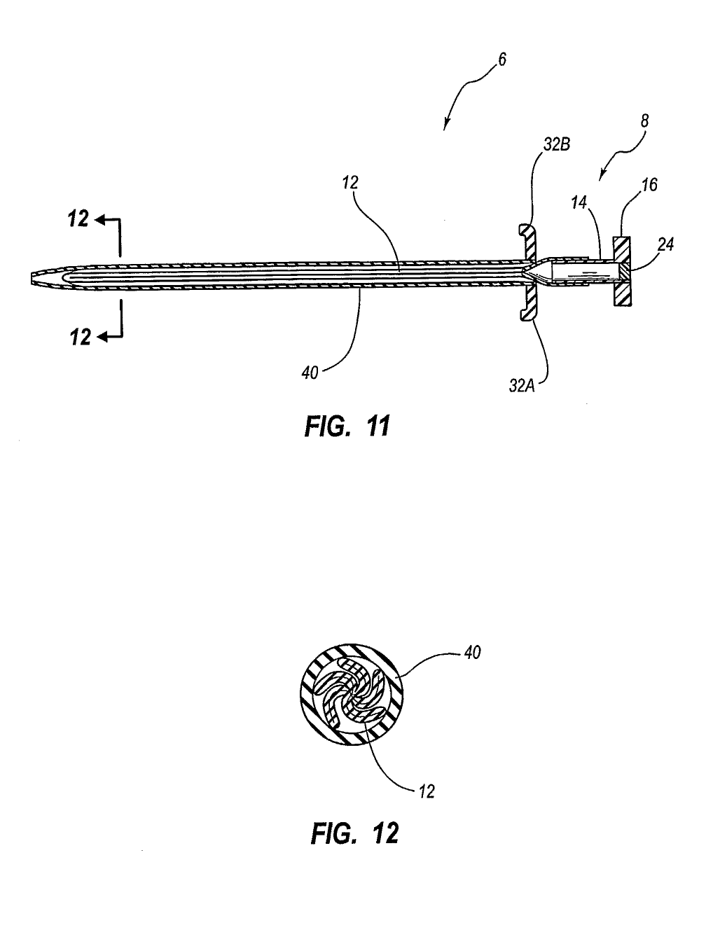

The present invention further contemplates that a gastric balloon device according to the present invention may ease the process by which such a device is positioned within and removed from a stomach of a patient. More particularly, relative to initial placement of a gastric balloon device within a stomach, the present invention contemplates that the at least one pleat of a balloon element may be furled or otherwise wrapped, rolled or folded about a central region thereof (i.e., about the longitudinal or axis of elongation thereof) so as to reduce the size of the balloon for insertion within a stoma formed through a stomach of a patient. Further, the present invention contemplates that a furled balloon element may be retained within a removable sleeve element during insertion thereof within a stomach of a patient. For example, FIG. 11 shows a side schematic view of one embodiment of a gastric balloon system 6 according to the present invention and configured for insertion within a patient. FIG. 12 shows a simplified cross-sectional view of the gastric balloon system 6 as shown in FIG. 11, the view taken along reference line C-C as shown in FIG. 11. Particularly, gastric balloon device 8 may be positioned within removable sleeve 40. It may be advantageous to generate a vacuum within chamber 18 of the balloon element 12 so as to reduce the size thereof for placing the balloon element 12 within the removable sleeve, as mentioned hereinbelow. Further, balloon element 12 may be furled, wrapped, rolled, or folded within removable sleeve 40. Such a configuration may provide a relatively compact gastric balloon system. In addition, removable sleeve 40 may be structured as a so-called split sleeve, wherein application of generally oppositely directed forces (i.e., away from shaft element 14) to tab structures 32A and 32B may cause the removable sleeve 40 to be severed into two substantially semicylindrical halves. In another embodiment, bolster 16 may not be initially coupled to shaft element 14 and the distal end of the removable sleeve may be open, so that upon positioning of the gastric balloon system 6 within a patient, the sleeve may slide longitudinally along the shaft element 14 and away from the balloon element 8, until it may be removed therefrom. Subsequent to removal of the removable sleeve 40, the bolster 16 may be coupled to the shaft element 14.

In further detail, a process for installing a gastric balloon system according to the present invention is outlined hereinbelow. Particularly, an insertion site may be selected and prepared and an incision may be formed so as to expose the stomach of a patient. Further, a needle or cannula may be inserted through the abdominal wall of the stomach and then may be removed. A dilator that is larger than the needle or cannula may be inserted into hole formed therewith to increase size of the opening. In addition, progressively larger dilator(s) may be employed until the opening is desirably sized. The last dilator may be positioned within the opening so that the gastric balloon system may be passed therethrough. Optionally, the removable sleeve 40 of the gastric balloon system 6 may be removed prior to placement through the dilator. In addition, the dilator may be a so-called split sheath dilator that is structured for splitting to form two substantially semicylindrical halves. A vacuum may be generated (e.g., by way of a syringe) within the balloon element so as to collapse the balloon element for ease of insertion within a stomach. Further, the distal end of the balloon element may be lubricated (e.g., with lubrication jelly) and inserted through the dilator split sheath until the balloon has passed completely into the stomach (i.e., as may be verified by endoscopic viewing). Also, the dilator split sheath may be removed by sliding it up the shaft element of the gastric balloon system while splitting the sheath in generally opposite directions (e.g., a tear-away sheath). The balloon element may then be expanded by introducing fluid therein. For example, a syringe having a specified amount of fluid (e.g., saline solution) may be inserted into the valve element and the fluid may be introduced into the balloon element.

As mentioned above, such a procedure may be verified by observation via an endoscope. The bolster may be adjusted as desired and positioning and size of the balloon element may be observed via an endoscope. In another embodiment, the balloon element may include a radiopaque tip (e.g., using barium sulfate or any other suitable radiopaque substance) to allow the position of the balloon to be determined within the stomach in order to verify that the desired placement has been made. In another suitable approach, the gastric balloon device may be filled with a so-called contrast medium (e.g., a solution including 2.5% barium, or any other contrast medium as known in the art) for exhibiting a perceivable level of radiopacity.

Explaining further, the gastric balloon device may be expanded or contracted based on individual needs (e.g., by introducing or removing fluid thereto or therefrom, respectively). Also, the gastric balloon device may be completely deflated for ease of removal and replacement. Moreover, subsequent to implantation, the gastric balloon device may be checked for leakage by removing the fluid therefrom and comparing the volume of fluid used to expand the gastric balloon device (e.g., via a syringe). Also, the fluid may be observed or otherwise tested for cloudiness, composition, or constituents, etc. that may indicate leakage. The above-described procedure for initial placement of the anti-obesity device is merely one illustrative procedure that may be used. It will be understood that any other suitable procedure may be used, that includes any, all, or none of the above actions in the same or different order for initial placement of the anti-obesity device.

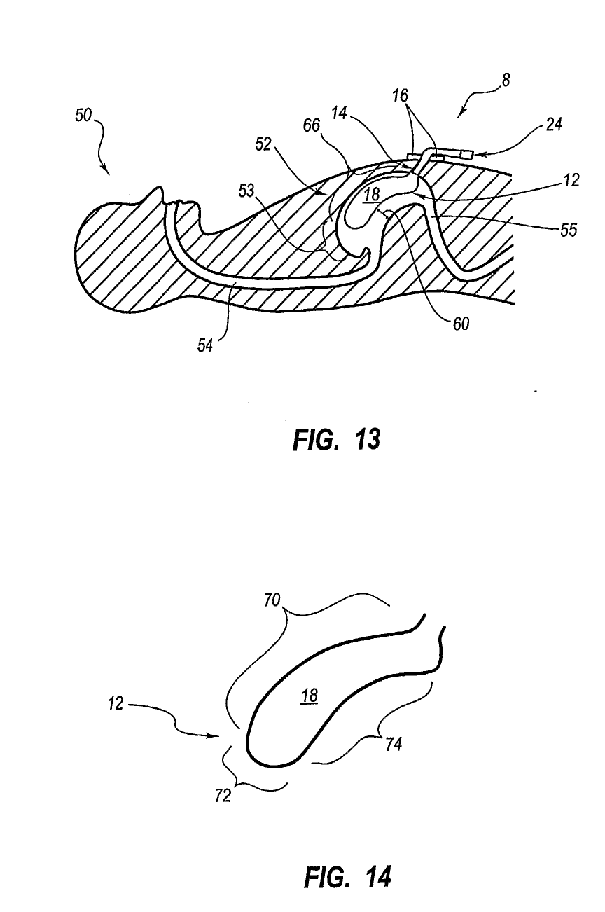

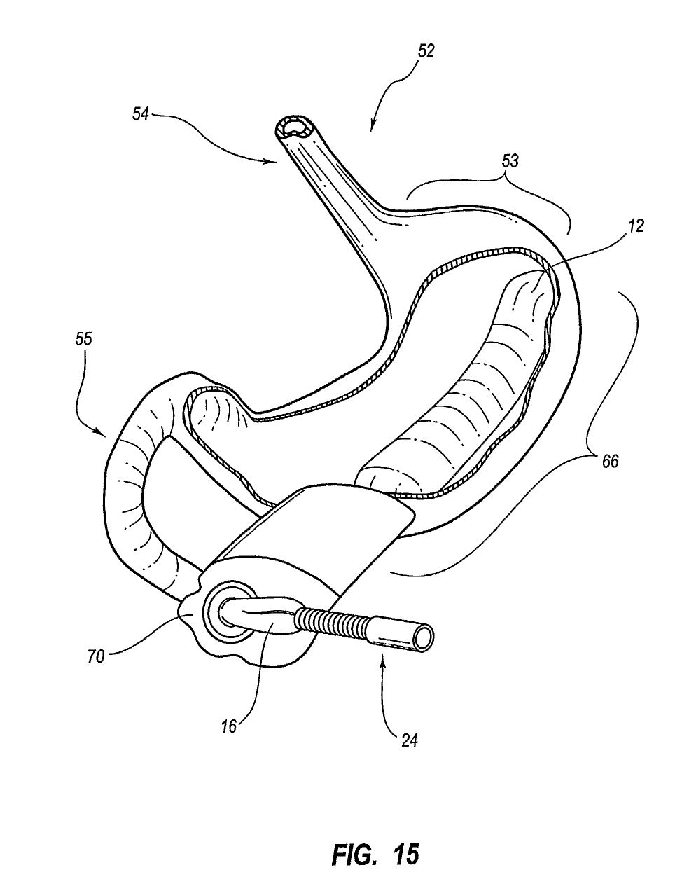

Thus, it may be appreciated that a gastric balloon device and system of the present invention may provide a less invasive alternative with fewer complications as compared to other conventional treatments of obese patients. Additional potential advantages may include lower cost, less trauma to the patient, shorter hospital stay, reduction in post-operation pain and recovery time, adjustable volume for individual needs (e.g., according to desired weight loss), procedure may be reversed (e.g., by removing the gastric balloon device). Generally, a gastric balloon device according to the present invention may be positioned within a stomach in any orientation that may be desired. In some embodiments of the present invention, the gastric balloon device may be anchored to the stomach wall and is positioned in such a way that prevents blockage of food passing therethrough. More particularly, in one embodiment, the present invention contemplates that a gastric balloon device of the present invention may be positioned so as to pass through a portion of the greater curvature of the stomach wall nearer to the duodenum than the fundus of the stomach. For example, FIG. 13 shows a schematic view of patient 50, esophagus 54 and stomach 52, wherein gastric balloon device 8 is positioned within stomach 52 of the patient 50. As shown in FIG. 13, shaft element 14 passes into the stomach 52 nearer to the duodenum 55 than the fundus region 53 of the stomach 52. Further, as shown in FIG. 13, balloon element 12 may be sized and configured so as to substantially conform to at least a portion of an interior of the greater curvature 66 of the stomach 52. Also, a gap or passageway 60 between the balloon element 12 and the lesser curvature of the stomach may be formed so as to allow passage of food therethrough. More particularly, FIG. 14 shows an enlarged view of FIG. 13, wherein balloon element 12 includes a convex region 70 (i.e., a convex region), a rounded end 72, and a concave region 74 (i.e., a concave surface). Such a convex region 70 may be positioned, sized, and configured for substantially conforming to at least a portion of a greater curvature region 66 of the stomach 50. Additionally, FIG. 15 shows a schematic cut-away perspective view of a gastric balloon device 8 implanted within a stomach 50 of a patient. As shown in FIG. 15, the bolster 16 may extend from the skin surface 70 to valve element 24. Further, as shown in FIG. 15, the gastric balloon device 8 is inserted through a portion of the greater curvature 66 of the stomach wall nearer to the duodenum 55 than the fundus region 53 of the stomach 50.

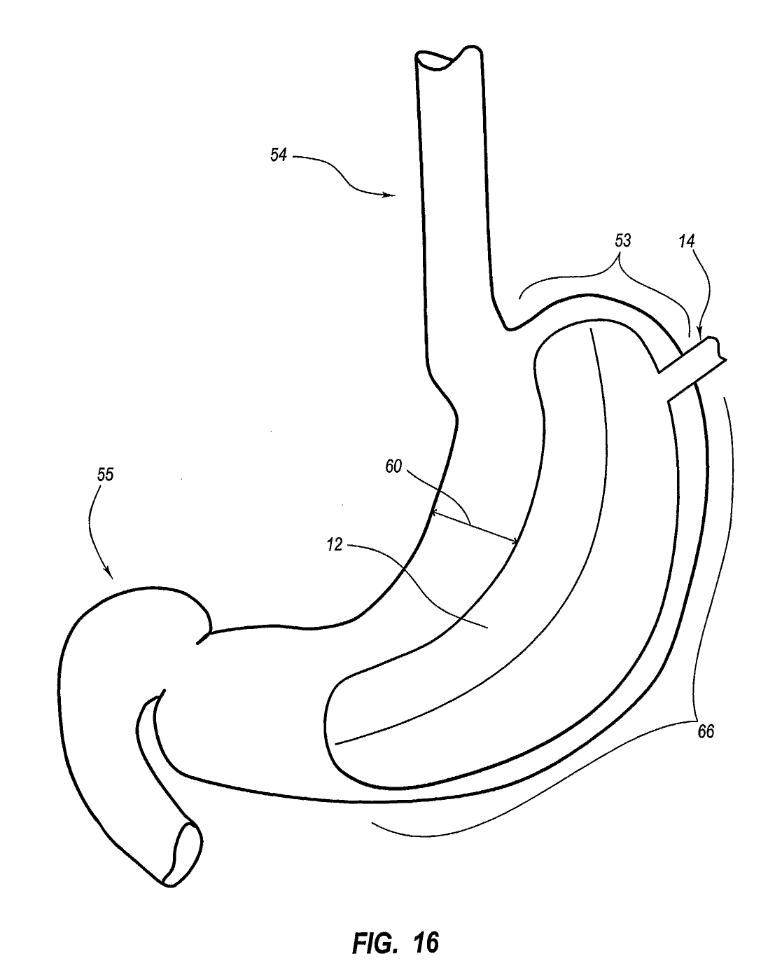

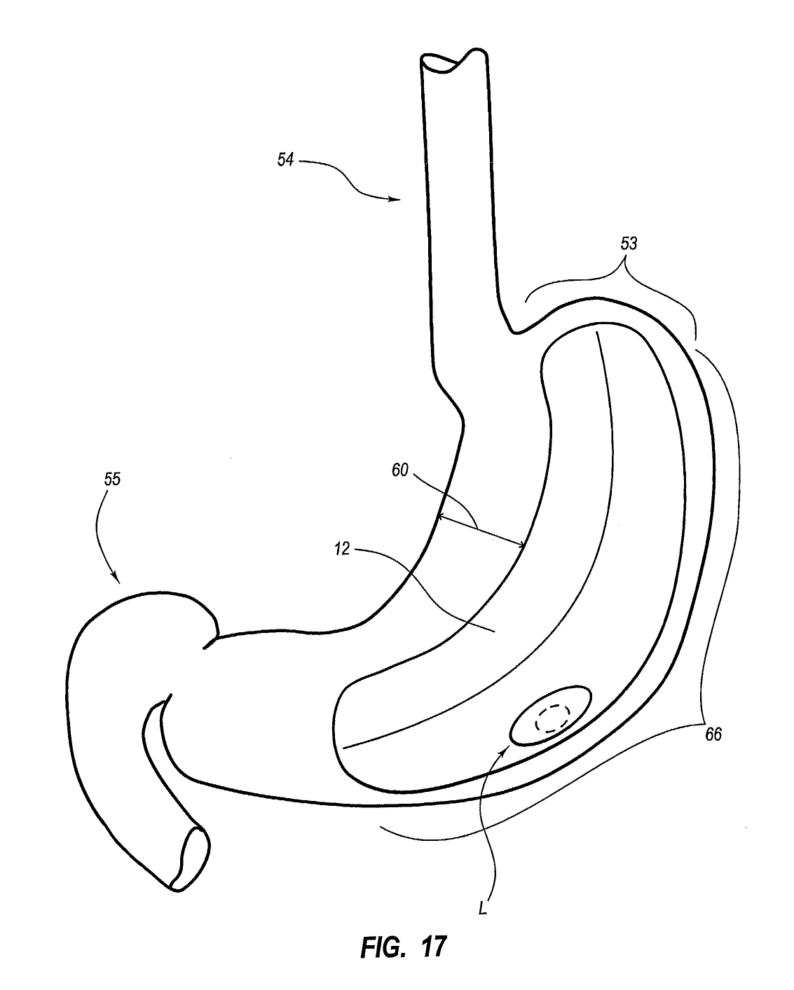

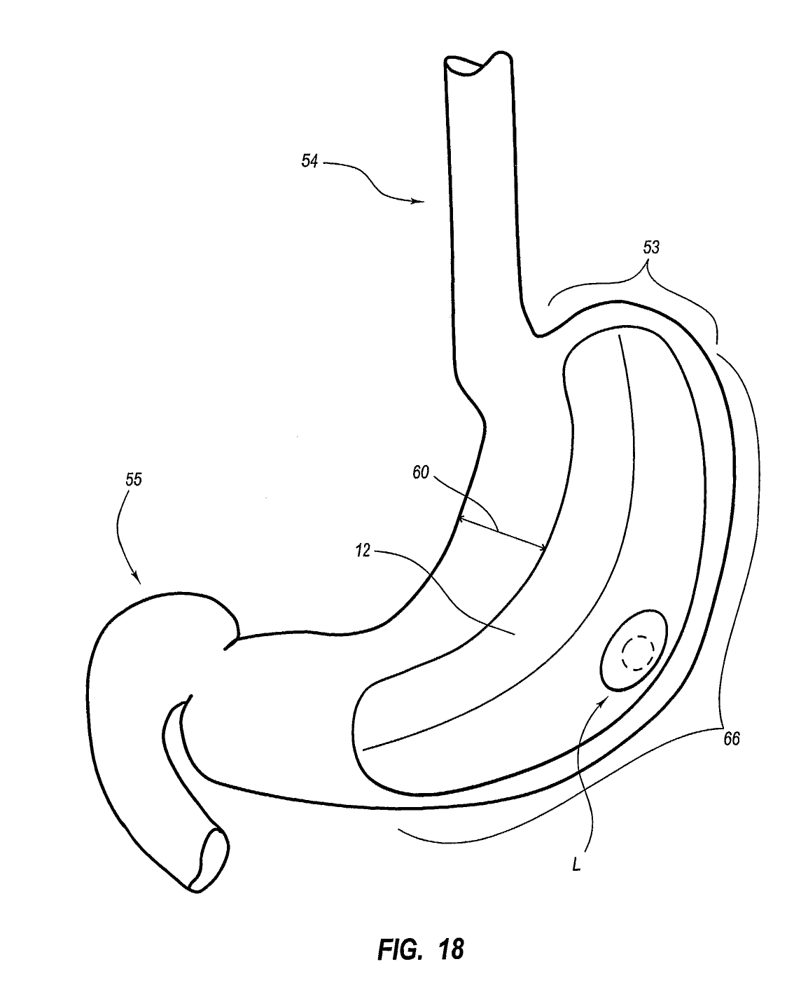

The present invention also contemplates that, in one embodiment, a gastric balloon device of the present invention may be positioned so as to pass through a portion of the greater curvature of the stomach wall nearer to a fundus region of the stomach than the duodenum. For example, as shown in FIG. 16, shaft element 14 passes into the stomach 52 nearer to the fundus region 53 than the duodenum 55 of the stomach 52. In addition, as shown in FIG. 16, balloon element 12 is sized and configured so as to substantially conform to at least a portion of an interior of the greater curvature 66 of the stomach 52. Also, a gap or passageway 60 between the balloon element 12 and the lesser curvature of the stomach may be formed so as to allow passage of food therethrough. However, the present invention contemplates other placements of a gastric balloon device according to the present invention. Particularly, in other embodiments, a gastric balloon device of the present invention may be inserted through a stoma that is located at any location along the greater curvature of the stomach, without limitation. For example, FIGS. 17 and 18 each show two different locations (labeled “L”) for a shaft element to pass through the stomach wall and which are generally centrally positioned with respect to the greater curvature 66 of the stomach 50. Thus, a gastric balloon device of the present invention may be positioned in a selected position within the stomach through a stoma formed along a selected position in the greater curvature of the stomach, without limitation.

The present invention further contemplates that a gastric balloon device according to the present invention may be employed for replacing another gastric balloon device. A process for removing a gastric balloon device according to the present invention is outlined hereinbelow. Initially, the fluid within the implanted gastric balloon device may be at least substantially removed therefrom (e.g., by inserting an empty syringe into the valve element and pulling the contents from the balloon element into the syringe). Optionally, a vacuum may be generated (e.g., by way of a syringe operably coupled to a valve element) within the balloon element so as to collapse the balloon element for ease of removal from the stomach. Such a vacuum may be maintained while removing the gastric balloon device from the stomach. Accordingly, a force may be applied to the gastric balloon device so as to pull it through the stoma formed into the stomach. Of course, another gastric balloon device of the present invention may be inserted into the existing stoma and expanded as described herinabove. It should be understood that after a stoma tract has been established within a patient, it may be desirable to employ a so-called “low profile” low profile bolster (e.g., a linear bolster or a 90° bolster), as known in the art.

While certain embodiments and details have been included herein for purposes of illustrating the invention, it will be apparent to those skilled in the art that various changes in the methods and apparatus disclosed herein may be made without departing form the scope of the invention, which is defined in the appended claims. Also, the words “including” and “having,” as used herein including the claims, shall have the same meaning as the word “comprising.”

What is claimed is:

1. A gastric balloon device comprising:
a shaft element for insertion through a stoma formed in a stomach of a patient; and
a generally elongated balloon element sealed to and extending from the shaft element and having at least one pleat region;
wherein at least a portion of the generally elongated balloon element is arcuately shaped.

2. The gastric balloon device of claim 1, wherein the balloon element is configured so as to, upon at least partial expansion, exhibit a concave region and a convex region.

3. The gastric balloon device of claim 1, wherein the convex region is sized and configured for substantially conforming to at least a portion of a greater curvature region of the stomach of the patient.

3. The gastric balloon device of claim 1, wherein the shaft element is sized and configured for extending through a portion of a greater curvature region of the stomach of the patient nearer to the duodenum then a fundus portion of the stomach so as to position the balloon element generally proximate to at least another portion of the greater curvature of the stomach.

4. The gastric balloon device of claim 1, wherein the balloon element includes a plurality of pleat regions.

5. The gastric balloon device of claim 4, wherein the balloon element includes four pleat regions arranged along substantially perpendicular axes.

6. The gastric balloon device of claim 4, wherein the balloon element includes three pleat regions arranged so as to form substantially equal angles therebetween.

7. The gastric balloon device of claim 1, wherein the plurality of pleat regions are furled.

8. The gastric balloon device of claim 1, further comprising a removable sheath positioned about the furled pleat regions of the balloon element.

9. The gastric balloon device of claim 1, further comprising a coating formed over at least a portion of the exterior of the balloon element.

10. The gastric balloon device of claim 9, wherein the coating is formed over substantially the entire exterior of the balloon element.

11. The gastric balloon device of claim 9, wherein the coating is formulated for exhibiting lubricity or for exhibiting chemical resistance to a chemical found in the stomach of the patient.

12. The gastric balloon device of claim 1, further comprising a bolster configured for retaining a position of the balloon element when positioned within the patient.

13. The gastric balloon device of claim 1, further comprising a valve element operably coupled to the balloon element and configured for controlling fluid communication with a chamber of the balloon element.

14. A gastric balloon device comprising:
a shaft element for insertion through a stoma formed in a stomach of a patient; and
a generally elongated balloon element sealed to and extending from the shaft element and having at least one pleat region.

15. A method of use of a gastric balloon system, the method comprising:
providing a gastric balloon system including a shaft element for insertion through a stoma formed in a stomach of a patient and a generally elongated balloon element sealed to and extending from the shaft element, wherein the balloon element includes at least one pleat region;
furling at least one pleat region of a balloon element of a gastric balloon device;
inserting the balloon element through a stoma into the stomach of the patient; and
expanding the balloon element.

16. The method of claim 15, wherein expanding the balloon element comprises introducing fluid within a chamber of the balloon element.

17. The method of claim 15, wherein introducing fluid within the chamber of the balloon element comprises introducing fluid within the chamber of the balloon element via a syringe.

18. The method of claim 15, further comprising positioning the balloon element within a removable sleeve subsequent to furling the at least one pleat region.

19. The method of claim 15, wherein inserting the balloon element through the stoma into the stomach of a patient comprises inserting the balloon element through a stoma formed nearer to a duodenum of the stomach than a fundus region of the stomach.

20. The method of claim 15, wherein expanding the balloon element comprises expanding the balloon element so as to substantially conform to at least a portion of a greater curvature region of the stomach of the patient.

21. A method of use of a gastric balloon system, the method comprising:
inserting a balloon element through a stoma into a stomach of a patient;
wherein the stoma is formed nearer to a duodenum of the stomach than a fundus region of the stomach; and
expanding the balloon element.

22. The method of claim 20, wherein inserting the balloon element through the stoma into the stomach of a patient comprises inserting the balloon element including at least one pleat region.

23. The method of claim 20, further comprising furling at least one pleat region of a balloon element of a gastric balloon device.

24. The method of claim 23, further comprising positioning the balloon element within a removable sleeve subsequent to furling the at least one pleat region.

25. The method of claim 21, wherein expanding the balloon element comprises introducing fluid within a chamber of the balloon element.

26. The method of claim 21, wherein introducing fluid within the chamber of the balloon element comprises introducing fluid within the chamber of the balloon element via a syringe.

27. The method of claim 21, further comprising, subsequent to expanding the balloon element, substantially removing the contents of the balloon element so as to collapse the balloon element.

28. The method of claim 27, wherein substantially removing the contents of the balloon element so as to collapse the balloon element comprises generating a vacuum within a chamber of the balloon element.

29. The method of claim 28, further comprising removing the balloon element from the stomach of the patient.
